(19) United States
(12) Patent Application Publication
(10) Pub. No.: US 2010/0100119 A1
(43) Pub. Date: Apr. 22, 2010

(54) MECHANICAL VEIN LIFTER

(76) Inventor: Terry O. Herndon, Carlisle, MA (US)

Correspondence Address: BANNER & WITCOFF, LTD., 28 STATE STREET, SUITE 1800, BOSTON, MA, 02109-1701, US

(21) Appl. No.: 12/253,317

(22) Filed: Oct. 17, 2008

(51) Int. Cl. A61B 17/00

(52) U.S. Cl. 606/201

(57) ABSTRACT

Devices for applying an intermittent pressure to a skin surface are provided. The device includes a housing, a drive mechanism at least partially disposed within the housing, and a pressing portion operatively coupled to the drive mechanism. The pressing portion may include at least one pressing element configured for applying pressure to the surface. The pressing portion is configured to be driven by the drive mechanism between a first configuration having a maximum extension and a second configuration having a minimum extension. The difference between the maximum and minimum extensions is greater than or equal to 0.025 inches. The pressing portion may include a series of rollers to press and squeeze the skin and underlying vein, to create a pumping action, thus pressurizing the vein. The vein expands and presses upward against the skin, thus becoming more visible.

### FIELD OF THE INVENTION

Disclosed is a mechanical device used to raise a vein in a body part in association with intravenous (IV) procedures, including, but not limited to, for example, drug delivery, catheterization, and blood drawing. This device automates the process of raising a vein by delivering a number of rapid, painless pressure pulses to the desired vascular access site.

### BACKGROUND OF THE INVENTION

Intravenous access, whether for drawing blood or for delivering fluids or drugs, is a necessary and unpleasant aspect of most medical diagnoses or treatments. However, for a variety of reasons, finding, raising and intravenously accessing a suitable vein may be very difficult, resulting in several attempts by medical personnel to establish access. This can result not only in considerable discomfort to the patient, but also in delays in establishing medical treatments.

Some hospital nursing staffs have specialists called “IV Nurses,” who are so designated because of their experience and skill in finding veins on the back of the hand into which they can place a hypodermic needle for infusion of fluids or withdrawal of analyte. In particular, the dorsal veins of the hand often are effectively invisible because of their small size, especially when they do not bulge up under the skin, as is the case of a vein in the inner fold of the elbow.

Traditionally a nurse will lightly tap or slap the skin of a patient at the desired venous access site to cause the vein to dilate, thereby rising up and becoming more visible and accessible. When the access site is on the hand or near the elbow, this process can be augmented by at least partially restricting the flow of blood downstream of the desired venous access site (for instance, by the application of a tourniquet) and/or by having the patient open and close their fist.

Another traditional method for augmenting the process of raising the vein is to apply heat to the desired venous access site, such as by placing a warm compress on the skin. A less common augmentation technique provides a continuous or pulsed suction action to the skin, using, for example, an air pump.

### PRIOR ART

US Patent Publication No. 2004/0171923 discloses a device to assist in gaining access to the vasculature of a patient that includes a vessel visualization device that assists a medical professional in finding the vessel and guiding a needle into the blood vessel. Further, the disclosed device may include a vessel immobilizer that keeps the vessel from moving under the skin by stretching the skin while applying pressure and/or trapping the vessel with a notched or pronged depressor.

US Patent Publication No. 2004/0267299 discloses a lancing device adapted to draw body fluid samples from the skin at a puncture site. This lancing device includes a vibration member to mechanically vibrate the skin adjacent to the puncture site during the lancing procedure. The vibration member is designed to reduce or eliminate pain associated with the lancing process. The vibration member may be a piezoelectric member. Alternatively, the vibration member may be a rigid member coupled via a cammed shaft to a motor or to an electrical outlet. The vibration member is placed in contact with, and may be pressed flush against, the patient's skin surface.

US Patent Publication No. 2005/0027218 discloses a device for applying a pulsating pressure to a local region of a body in order to increase blood flow in that region. The device includes a pressure chamber partially filled with liquid into which the limb of the patient can be placed. Negative pressure pulses can be generated in the liquid, for example, by a pump or a vacuum line in conjunction with a regulating device.

US Patent Publication No. 2007/0088385 discloses a vibrating tourniquet to decrease the sensation of pain when a needle or IV is introduced through the skin and into a vein. Vibration devices, which are attached to a flexible strap, are coupled to a controller and an electrical power source. The controller may allow the vibration devices to vibrate at a variety of different amplitudes and frequencies.

U.S. Pat. No. 3,620,209 discloses a vibrating device adapted to be placed on the skin of a patient to stimulate the pain center of the skin during an injection so as to reduce or eliminate the pain normally associated with an injection. A casing is strapped to the patient and caused to vibrate against the patient's skin while allowing access to the injection site.

U.S. Pat. No. 4,834,802 discloses a heat generating tourniquet for venipuncture applications. The tourniquet includes a heating pad having a window. When the pad is tightly secured to a patient with the window positioned over a blood vessel, a portion of the patient's skin is mechanically puffed up through the window and the blood vessel is mechanically secured. The blood vessel is also locally vasodilated.

U.S. Pat. No. 5,312,350 discloses a vein spotter which consists of a flexible band with a plurality of protrusions located on the inside of the band. When the band is placed around a limb, the protrusion members push into the skin, displacing tissue and causing superficial veins to distend and become visible.

U.S. Pat. No. 6,231,531 discloses an apparatus for minimizing pain perception during an invasive medical procedure. A vibratory mechanism transmits vibrations through a receiving surface to a patient's skin while a sharp extends through the receiving surface to puncture the patient's skin. The vibratory mechanism may include weighted cams or eccentrics associated with a motor for movement of the receiving surface in the plane of the receiving surface. The vibratory mechanism may also include an electromagnet placed between two permanent magnets or a roughened roller associated with actuation of the sharp.

None of the art cited above either teaches or suggests a mechanical vein massager or dilator device as taught herein. There are a number of devices that vibrate the skin—but they do so as a pain reduction technique—with the ongoing vibrations either canceling or overwhelming the pain signals when the skin is punctured.

What is needed is a device to automate the process of raising or dilating a vein in a body part to facilitate locating and puncturing the vein more efficiently, with greater accuracy, and with less patient discomfort than is currently possible, and for facilitating the insertion of a hypodermic needle into the desired vein for introducing drugs into the body, withdrawing body fluids, or affixing an intravenous port for longer term fluid supply to the body.

### SUMMARY OF THE INVENTION

Certain embodiments of a device for applying intermittent pressure to a skin surface are described. Such devices press and squeeze the skin and underlying vein, creating a pumping action, thereby pressurizing the vein. Thus, the vein expands and presses upwardly against the skin, becoming more visible and accessible.

In accordance with a first aspect, a device for applying an intermittent pressure to a surface includes a housing, a drive mechanism at least partially disposed within the housing, and a pressing portion operatively coupled to the drive mechanism. The pressing portion includes at least one pressing element configured for applying pressure to the surface. The pressing portion is configured to be driven by the drive mechanism between a first configuration having a maximum extension and a second configuration having a minimum extension. A difference between the maximum and minimum extensions is greater than or equal to 0.025 inches.

In accordance with another aspect, a device for applying an intermittent pressure to a skin surface includes a housing, a drive mechanism at least partially disposed within said housing, and a pressing portion coupled to the drive mechanism. The pressing portion has at least one pressing surface configured for applying pressure to the skin surface. The pressing portion rotates relative to the housing.

In accordance with a third aspect, a method for accessing a vein is provided. The method includes positioning a pressing portion of a pressing mechanism over a skin surface; intermittently applying pressure to the skin surface with the pressing portion; stopping the intermittent application of pressure to the skin surface; and thereafter puncturing the skin surface.

The pressing portion may intermittently apply pressure at a frequency of from approximately 2 Hz to approximately 25 Hz. Further, the pressing portion may be configured to depress the skin by greater than or equal to 0.025 inches.

Moving the pressing portion may include oscillating the pressing portion relative to the housing of the pressing mechanism. A displacement magnitude of the oscillations may be greater than approximately 0.025 inches. In one embodiment, the displacement magnitude of the oscillations may range from approximately 0.08 inches to approximately 0.13 inches. Further, the pressing mechanism may be slid substantially along the longitudinal axis of the vein.

Features and advantages disclosed herein will be further understood from the following detailed disclosure of certain embodiments.

### BRIEF DESCRIPTION OF THE DRAWINGS

FIG. 1 illustrates a side view of a first embodiment of a device for applying an intermittent pressure to a skin surface, with the interior of the housing exposed;

FIG. 2A shows the embodiment of FIG. 1 in a first configuration;

FIG. 2B show the embodiment of FIG. 1 in a second configuration;

FIG. 3 illustrates a side view of a second embodiment of a device for applying an intermittent pressure to a skin surface; and

FIG. 4 shows an end view of the embodiment of FIG. 3 in a second configuration.

The figures referred to above are not drawn necessarily to scale and should be understood to provide a representation of a device for applying an intermittent pressure to a skin surface. Some features of the device depicted in the drawings may have been enlarged or distorted relative to others to facilitate explanation and understanding. The same reference numerals are used in the drawings for similar or identical components and features shown in various alternative embodiments. A device for applying an intermittent pressure to a skin surface, as disclosed herein, would have configurations and components determined, in part, by the intended application and environment in which it is used, as would be apparent to persons of ordinary skill in the art, given the benefit of this disclosure.

### DETAILED DESCRIPTION OF THE PREFERRED EMBODIMENT

As illustrated in the drawings accompanying this disclosure, the present application is directed to a device that causes veins to protrude upwardly under the skin surface, for example on the back of the hand, or elsewhere. The device automates the process of raising a vein in a body part in order to facilitate locating and puncturing the vein as is required for delivering intravenous procedures. In one aspect, the device automates that process by delivering a number of pressure pulses to the desired site, thereby causing the vein to rise.

A device for causing a vein to become more prominent and accessible may be embodied in various forms. In one aspect, the disclosed device causes a vein to elevate and be more visible simply by pulsating up and down on the surface of the skin above the vein, which compresses and decompresses the vein under the skin surface. This tends to pressurize the vein under the skin, causing the vein to dilate or “swell up.” The vein rises up under the surface of the skin enough to make it both more visible and more accessible for access by a needle.

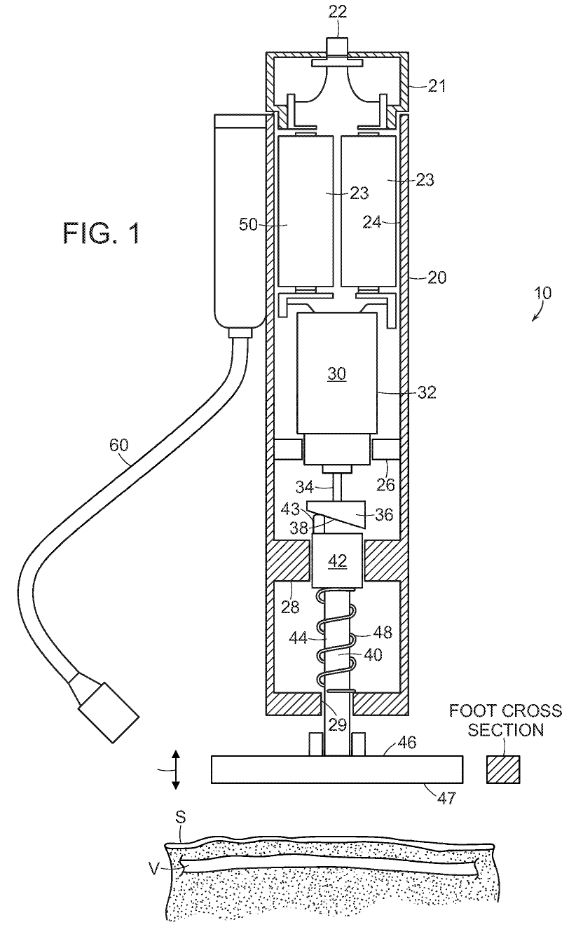

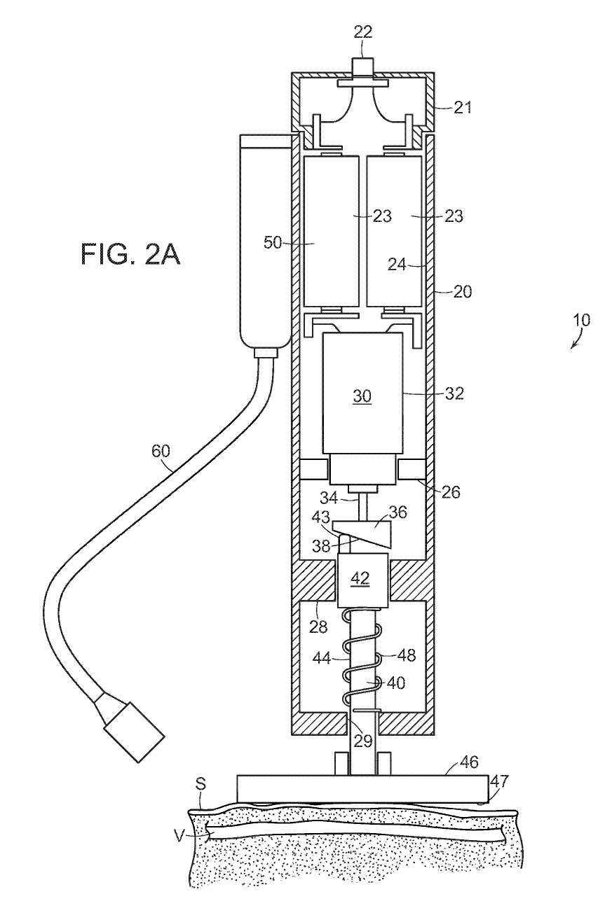

FIG. 1 illustrates a first embodiment of the device described in this application. FIG. 2A illustrates the embodiment of FIG. 1 in a first configuration, with the device just contacting a skin surface. FIG. 2B illustrates the embodiment of FIG. 1 in a second configuration, with the device applying pressure to a skin surface.

Referring to FIG. 1, a device for applying an intermittent pressure to a skin surface (S) is provided. Intermittent pressure device 10 includes a housing 20, a drive mechanism 30 and a pressing portion 40. Device 10 may further include a power source 50. A light source 60 may also be provided with device 10. Device 10 may be portable. Further device 10 may be of a suitable weight and size to allow it to be handheld.

In this first embodiment, housing 20 is substantially cylindrical. Housing 20 includes a removable end cap 21 at a first end. A switch 22 for turning drive mechanism 30 on and off is located at the top of housing 20, within end cap 21. Switch 22 is shown as a push switch. A battery compartment 24 is located beneath switch 22 and is configured to house power source 50, which, in this particular embodiment, includes two AA batteries 23. Drive mechanism 30 is located within housing 20 beneath battery compartment 24. Motor mount 26 securely holds drive mechanism 30 to the outer wall of housing 20. Pressing portion 40, located partially within housing 20, beneath drive mechanism 30, extends out through a hole 29 in the bottom end of housing 20. Pressing portion mount 28 slidably secures pressing portion 40 to housing 20.

Drive mechanism 30 is at least partially disposed within housing 20. In the embodiment of FIGS. 1, 2A and 2B, drive mechanism includes a gear motor 32, a rotational shaft 34 extending from gear motor 32 and a cam 36 coupled to the end of shaft 34 opposite the end coupled to gear motor 32. Cam 36 is provided with a surface 38 that is oriented at a non-perpendicular angle to the longitudinal axis of shaft 34. Drive mechanism 30 may be of any suitable configuration, as would be appreciated by a person of ordinary skill in the art given the benefit of this disclosure. Drive mechanism 30 may be powered by batteries stored within housing, by an external power source, or by any suitable power source, as would be apparent to a person of ordinary skill in the art given the benefit of the present disclosure.

Pressing portion 40 is operationally coupled to drive mechanism 30. Pressing portion 40 includes a cam follower 42 in the form of an output plunger. Rod 43 of cam follower 42 rides up and down on the slanted or inclined surface 38 of cam 36 when gear motor 32 causes rotational shaft 34 to spin. Pressing portion 40 further includes a shaft 44 that extends from cam follower 42, through the bottom end of housing 20 to a pressing element 46. Pressing element 46 has a pressing surface 47 for contacting skin surface (S). Pressing portion 40 further includes a spring 48 for biasing pressing element 46 away from the skin surface (S). Other mechanisms for operationally coupling pressing portion 40 to drive mechanism 30 would be apparent to a person of ordinary skill in the art given the benefit of this disclosure.

Pressing element 46 is configured to apply pressure to the skin surface, such that a vein (V) located below the skin surface will be compressed. The pressing surface 47 of pressing element 46 may be circular, rectangular, or any other suitable shape. Further, pressing surface 47 may have softened or rounded edges such that the edges do not dig into the skin surface. Even further, pressing surface 47 need not be flat, but could have a contoured, three-dimensional shape-such as a wedge, one portion touching first, going deeper as the remainder touches, or producing a squeezing or pumping action. By way of non-limiting examples, a pressing or contact area could be approximately 0.5 inches by approximately 0.125 inches rectangular. It is expected that a pressing area of approximately 0.05 square inches to approximately 0.20 square inches would be most suitable. Although the shape of the contact area is not critical, in one embodiment, at least one skin-contacting dimension of pressing surface 47 is approximately 0.4 inches or greater.

Pressing element 46 may be formed of a relatively rigid material, such as a metal or a plastic material, or of a less rigid material, such as rubber or an elastomeric material. In one embodiment, pressing element 46 may be formed of more than one material. For example, a more rigid material may form a platform upon which a less rigid, skin-contacting material may be applied. In another exemplary embodiment, a thin skin-contacting material or sheath may be disposably or replaceably placed over pressing surface 47 for hygienic purposes.

As best shown in FIGS. 2A and 2B, pressing portion 40 is configured to be driven between a first configuration associated with a minimum extension (FIG. 2A) and a second configuration associated with a maximum extension (FIG. 2B). Referring to FIG. 2A, in the first configuration, cam 36 is rotated such that rod 43 of cam follower 42 is positioned at the top of the inclined portion 38 of cam 36. Spring 48 biases cam follower 42 against this inclined portion. In other words, pressing portion 40 is maximally pushed up into housing 20. In this configuration, deflection of the skin surface and any compression of an underlying vein would be at a minimum. Referring to FIG. 2B, in the second configuration, cam 36 is rotated such that rod 43 of cam follower 42 is positioned at the bottom of the inclined portion 38 of cam 36. In other words, in the second configuration pressing portion 40 is maximally extended out of housing 20. In this configuration, deflection of the skin surface and the corresponding compression of any underlying vein would be at a maximum.

In operation, as drive mechanism 30 causes cam 36 to rotate, rod 43 and cam follower 42 slide up and down along the inclined surface 38 of cam 36, thus causing pressing portion 40 to linearly oscillate relative to housing 20 between a minimum and a maximum extension. The difference (Δ) between the minimum and maximum extension of pressing portion 40 may range from on the order of 0.025 inches to on the order of 0.13 inches. The smaller difference in extension may be particularly applicable for children or the elderly; the larger difference in extensions may be particularly applicable for large men or obese patients. Thus, by way of non-limiting examples, a difference in extension (Δ) of between 0.025 and 0.09 inches may be particularly applicable for children; while a difference in extension (Δ) of between 0.08 and 0.13 inches may be particularly applicable for most adults. In one embodiment, a mechanism or switch for limiting the maximum extension may be provided, such that a single device 10 could provide linear oscillations of varying magnitudes.

In one embodiment, the frequency of the linear oscillations of pressure portion 40, i.e. the frequency of the intermittently applied pressure, may be from approximately 2 Hz to approximately 25 Hz. A mechanism or switch for either increasing or decreasing the frequency may be included in the device, such that a health care worker may tailor the application to the individual patient. By way of non-limiting examples, the health care worker may set the frequency in a range of from approximately 5 Hz to approximately 20 Hz, within a narrower range of from approximately 5 Hz to approximately 15 Hz, within a relatively low frequency range of from approximately 2 Hz to approximately 12 Hz, or within a relatively high frequency range of from approximately 15 Hz to approximately 25 Hz.

As shown in FIGS. 1, 2A and 2B, pressing device 10 may further include a light 60. In one embodiment, light 60 is a small, bright light adjustably aligned to shine in a grazing or near-grazing fashion across the skin in the assumed path of the vein. Light 60 may be detachably attached to housing 20 and powered from the same batteries 23 that power motor 30.

Thus, by way of non-limiting example, a hand held device may be provided for intermittently applying pressure to a skin surface and an underlying vein. The device may include a battery power supply, a small electric motor that drives a gear train or a cam system to move a shaft of a pressing portion back and forth (in and out) through the end of the device nearest the skin. The pressing portion may have a pressing surface with a skin contacting area of from approximately 0.05 to 0.2 square inches. The linear stroke of the shaft may range from approximately 0.025 inches to 0.13 inches. The frequency of the linear stroke may be typically between 2 cycles/second and 25 cycles/second. For example, the device may pulsate at about 10 pulses per second and move up and down about a tenth of an inch.

Without limiting the device in any manner, it is believed that the device functions because veins contain one-way valves that permit the blood to flow in one direction and not the other. Pressing on a vein compresses it shut, causing blood to be pumped (squeezed out of) away from the collapsed area to be blocked by a valve in one direction and to flow through the valve on the other side. The intermittent application of pressure causes the vein to overfill with blood and to expand somewhat, thereby pushing the vein's upper surface closer to the back of the skin. In operation, it may be beneficial to move the device parallel to the surface of the skin, along or near the path or the longitudinal axis of the vein, during the application of the intermittent pressure. Even further, a potentially more efficient way to raise a vein would be to slide the vibrating device along the vein path away from the heart, lifting the device off of the skin at the end of the sliding movement, placing the device back on the skin along the vein toward the heart, pressing the device against the skin and sliding it away from the heart again. The distance of sliding movement is determined by experience with particular locations, but is expected to be typically on the order of 0.5 inches to 3 inches at a rate of approximately 1-2 seconds per slide. Under the application of the intermittent pressure, a vein may become visible within 10 to 15 seconds, with a maximum lifting/swelling of the vein possibly occurring within 30 seconds. This lifting of the vein is expected to persist for 10 to 20 seconds after the cessation of the application of intermittent pressure-long enough for a health care professional to align and insert a hypodermic needle.

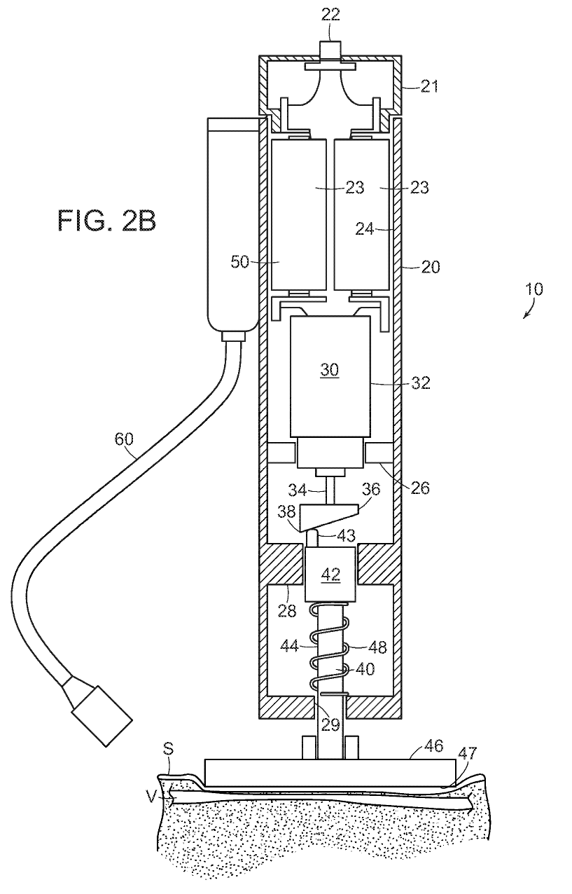

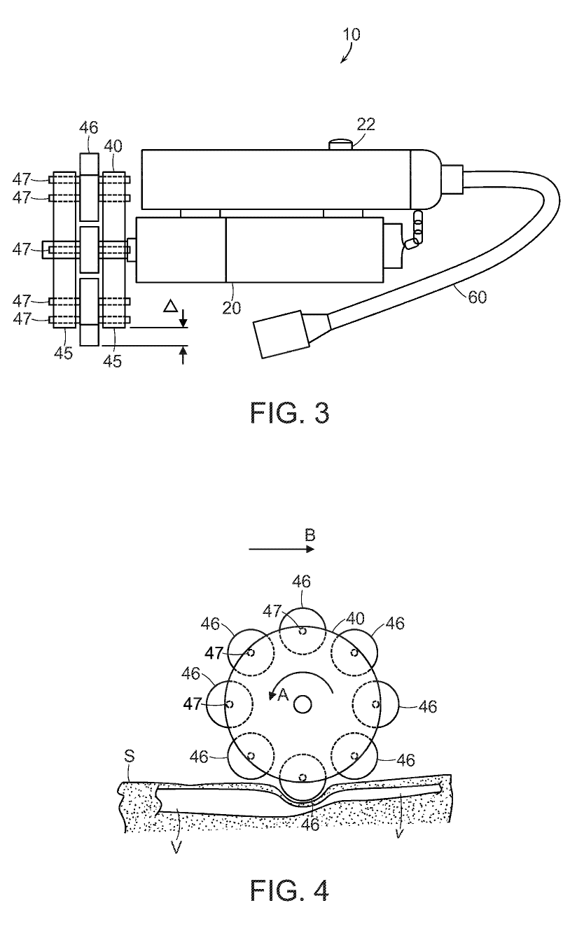

Referring to FIGS. 3 and 4, another embodiment of a device 10 for intermittently applying pressure to a skin surface is illustrated. Device 10 includes a housing 20, a rotary drive mechanism at least partially disposed within the housing and a pressing portion 40 operatively coupled to the drive mechanism. In this embodiment, pressing portion 40 is rotatably driven by drive mechanism relative to housing 20 in the direction of arrow A (see FIG. 4).

Pressing portion 40 includes two wheels 45 separated from each other by a width suitable for accommodating pressing elements 46. In this particular embodiment, eight pressing elements 46 are arranged around the periphery of wheels 45. By way of non-limiting examples, the diameter of wheels 45 may range from approximately ¾ of an inch up to 3 inches and the separation width between the wheels may range from approximately a tenth to a quarter of an inch. As best shown in FIG. 4, pressing elements 46 may be small circular elements coupled to and extending beyond the periphery of wheels 45. Pressing elements 46 may be rotatably coupled to wheels 45 via axles 47. By way of a further non-limiting example, pressing elements 46 may be approximately an eighth inch wide and have a diameter ranging from a quarter to three-eighths of an inch. Variations of the pressing portion are within the scope of the invention. As a non-limiting example, pressing portion 40 may be a unitary wheel having raised peripheral portions, somewhat like a cog. As other non-limiting examples, pressing portion 40 may include a resilient element for limiting the pressure applied to the skin surface, or alternatively, the individual pressing elements could be spring-loaded. Other embodiments of a rotatable pressing portion would be readily apparent to a person of ordinary skill in the art given the benefit of the present disclosure.

In operation, pressing portion 40 is turned by a small electric motor powered by batteries or other power supply. A shaft and/or gear train operatively couples pressing portion 40 to the motor. A first pressing element 46 is held lightly against the skin and moved or rolled along the presumed vein path. In this case, the pressing element touching the skin (S) compresses the vein (V) as the pressing element comes into contact with the skin surface. As the pressing portion continues to rotate, the pressing element continues to move the blood along the compressed vein toward a vein valve (v) until the pressing element lifts away. Meanwhile, a second, following pressing element begins to touch the skin, trapping blood in the vein ahead of its motion and moving the blood in the direction of the pressing portion motion. Thus, this system functions somewhat like a peristaltic pump, a well-known device that moves fluids in compressible tubing without touching the fluid. Additionally, while operating, the entire device 10 may be moved in a direction of arrow B (see FIG. 4, away from the heart), in order to facilitate the movement of blood within the vein during operation.

According to one aspect of the invention, during operation of the device 10, it may be desirable for an operator or other health care worker to press down on the venous area that lies to the heart side of vein portion being intermittently compressed. This causes constriction or quasi-constriction of the venous area. This could be accomplished, for example, by the operator gripping the wrist of a patient while applying device 10 to a vein portion on the back of the hand. Device 10 is slid toward the patient's heart and toward the gripped area at the wrist along the back of the hand. This “pumps” blood through the valves in the vein against the constriction or quasi-constriction formed by the operator's grip, allowing the vein to inflate.

As the drive mechanism causes pressing portion 40 to rotate relative to housing 20, the skin surface is depressed between a maximum and a minimum. The maximum depression of the skin surface is associated with a maximum extension of the pressing portion, which occurs when a pressing element is centered beneath the axis of rotation of the pressing portion. The minimum depression of the skin surface is associated with a minimum extension of the pressing portion, which occurs when a point midway between two adjacent pressing elements is centered beneath the axis of rotation of the pressing portion. Thus, in this embodiment, the difference (Δ) between the maximum extension and the minimum extension is the distance that the pressing element 46 extends beyond the periphery of the wheels 45 (see FIG. 3). In general, a suitable difference (Δ) between the minimum and maximum extension of pressing portion 40 may range from on the order of 0.025 inches to on the order of 0.13 inches. By way of non-limiting examples, a difference in extension (Δ) of between 0.025 and 0.09 inches may be particularly applicable for children; while a difference in extension (Δ) of between 0.08 and 0.13 inches may be particularly applicable for most adults. In one embodiment, different pressing portions 40, having any of various differences (Δ) between the minimum and maximum extensions, may be interchangeably and replaceably coupled to the end shaft of the drive mechanism.

As with the embodiment of FIGS. 1, 2A and 2B, a typical frequency for the application of the maximum intermittently applied pressure, may be from approximately 2 Hz to approximately 25 Hz. These frequencies can be set by varying the rotational speed of the drive mechanism. A mechanism or switch for either increasing or decreasing the frequency may be included in the device, such that a health care worker may tailor the application to the individual patient.

A light 60, optionally with its own battery pack, may be built into the system to provide grazing light across the skin to help highlight the expanded and lifted vein.

By way of explanation and without limiting the device in any manner, it is believed that the device takes advantage of the fact that veins have valves in them to help return blood to the heart by preventing it from flowing back to the lower extremities due to gravity. In this embodiment, the vein (V) acts as a compressible tube and the rotatably pressing elements 46 compress the vein through the skin (S) and a thin layer of the epidermis. The rotation of the pressing portion 40 pumps the fluid toward the vein's valve (v) while the rotating pressing elements 46 move along the skin. This combined action serves to pump more blood into the vein (V) against the valve (v) which serves to temporarily pressurize the vein and cause it to expand. Since most veins, for example, those in the back of the hand or wrist, are relatively near the outside surface of the skin, the result is that the increased diameter causes the vein to press outward against the back of the skin, causing the vein to be easily seen.

The present application has described a method for accessing a vein and a device that causes a vein to elevate and be more visible by intermittently pressing on the surface of the skin above the vein to pump the vein under the skin surface and/or rolling on the surface of the skin above the vein to prevent or hinder the natural flow of the blood in the vein. This tends to cause the vein to swell or dilate, thus making it rise up or lift under the skin, becoming more visible and more accessible for intravenous access.

As described above, one preferred embodiment of the present invention is a method for accessing a vein, comprising:
positioning a pressing portion of a pressing mechanism over a skin surface;
intermittently applying pressure to the skin surface with the pressing portion;
stopping the intermittent application of pressure to the skin surface; and thereafter puncturing the skin surface.
   Preferably the method of applying pressure includes moving said pressing portion relative to a housing of said pressing mechanism. Preferably, the pressing portion intermittently applies pressure at a frequency of from approximately 2 Hz to approximately 25 Hz. Preferably, the pressing portion is configured to depress the skin by greater than or equal to 0.025 inches. Preferably, the pressing portion includes oscillating said pressing portion relative to said housing of said pressing mechanism. Preferably, the displacement magnitude of the oscillations is greater than approximately 0.025 inches. More preferably, the displacement magnitude of the oscillations ranges from approximately 0.08 inches to approximately 0.13 inches. Alternatively, the pressing portion includes rotating said pressing portion relative to said housing of said pressing mechanism. Advantageously, the pressing portion includes a plurality of pressing surfaces. Further, the method may include sliding said pressing portion substantially along the longitudinal axis of the vein.

As described above, one preferred embodiment of the present invention is a device for applying an intermittent pressure to a surface, the device comprising:
a housing;
a drive mechanism at least partially disposed within said housing; and
a pressing portion operatively coupled to said drive mechanism,
   said pressing portion including at least one pressing element configured
for applying pressure to the surface,
   said pressing portion configured to be driven by said drive mechanism
between a first configuration having a maximum extension and a second configuration having a minimum extension, and
   wherein a difference between said maximum and minimum extensions is greater than or equal to 0.025 inches.
      Preferably, the difference between the maximum and minimum extensions is greater than or equal to 0.08 inches. More preferably, the difference is between approximately 0.08 inches and approximately 0.13 inches. Advantageously, the pressing portion linearly oscillates relative to said housing between said maximum and minimum extensions. Alternatively, the pressing portion rotates relative to said housing. Advantageously the pressing portion includes a plurality of pressing elements arranged on a periphery of said pressing portion. Preferably the pressing element has a pressing area of approximately 0.05 square inches to approximately 0.20 square inches. Advantageously, the device further includes a light source coupled to said housing.

As described above, one preferred embodiment of the present invention is a device for applying an intermittent pressure to a skin surface, the device comprising:
a housing;
a drive mechanism at least partially disposed within said housing; and
a pressing portion coupled to said drive mechanism,
   said pressing portion having at least one pressing surface configured for
applying pressure to the skin surface,
   wherein said pressing portion rotates relative to said housing.
      Preferably the pressing portion includes a plurality of pressing elements arranged on a periphery of said pressing portion. More preferably, the pressing elements are movably incorporated into said pressing portion.

In light of the foregoing disclosure and description of various embodiments, those skilled in the area of this technology will readily understand that various modifications and adaptations can be made without departing from the scope and spirit of the invention.

What is claimed is:

1. A method for accessing a vein, comprising:
positioning a pressing portion of a pressing mechanism over a skin surface;
intermittently applying pressure to the skin surface with the pressing portion;
stopping the intermittent application of pressure to the skin surface; and thereafter puncturing the skin surface.

2. The method of claim 1, wherein intermittently applying pressure includes moving said pressing portion relative to a housing of said pressing mechanism.

3. The method of claim 1, wherein said pressing portion intermittently applies pressure at a frequency of from approximately 2 Hz to approximately 25 Hz.

4. The method of claim 1, wherein said pressing portion is configured to depress the skin by greater than or equal to 0.025 inches.

5. The method of claim 2, wherein moving said pressing portion includes oscillating said pressing portion relative to said housing of said pressing mechanism.

6. The method of claim 5, wherein a displacement magnitude of said oscillations ranges from approximately 0.025 inches to approximately 0.13 inches.

7. The method of claim 2, wherein moving said pressing portion includes rotating said pressing portion relative to said housing of said pressing mechanism.

8. The method of claim 2, wherein said pressing portion includes a plurality of pressing surfaces.

9. The method of claim 1, further comprising sliding said pressing mechanism substantially along the longitudinal axis of the vein.

10. A device for applying an intermittent pressure to a surface, the device comprising:
a housing;
a drive mechanism at least partially disposed within said housing; and
a pressing portion operatively coupled to said drive mechanism,
said pressing portion including at least one pressing element configured for applying pressure to the surface,
said pressing portion configured to be driven by said drive mechanism between a first configuration having a maximum extension and a second configuration having a minimum extension, and
wherein a difference between said maximum and minimum extensions is greater than or equal to 0.025 inches.

11. The device of claim 10, wherein said difference is greater than or equal to 0.08 inches.

12. The device of claim 10, wherein said difference is between approximately 0.08 inches and approximately 0.13 inches.

13. The device of claim 10, wherein said pressing portion linearly oscillates relative to said housing between said maximum and minimum extensions.

14. The device of claim 10, wherein said pressing portion rotates relative to said housing.

15. The device of claim 10, wherein said pressing portion includes a plurality of pressing elements arranged on a periphery of said pressing portion.

16. The device of claim 10, wherein said pressing element has a pressing area of approximately 0.05 square inches to approximately 0.20 square inches.

17. The device of claim 10, further including:
a light source coupled to said housing.

18. A device for applying an intermittent pressure to a skin surface, the device comprising:
a housing;
a drive mechanism at least partially disposed within said housing; and
a pressing portion coupled to said drive mechanism,
said pressing portion having at least one pressing surface configured for applying pressure to the skin surface,
wherein said pressing portion rotates relative to said housing.

19. The device of claim 18, wherein said pressing portion includes a plurality of pressing elements arranged on a periphery of said pressing portion.

20. The device of claim 19, wherein said pressing elements are movably incorporated into said pressing portion.
